(19) United States
(12) Plant Patent Application Publication
(10) Pub. No.: US 2010/0138970 P1
(43) Pub. Date: Jun. 3, 2010

(54) Ornithogalum plant named 'Damascus'

(50) ;

(76) Inventor: Rodolfo Valdoz Bautista, Half Moon Bay, CA (US)

Correspondence Address: Law offices of James R. Cypher, 409 13th Street, 11th Floor, OAKLAND, CA, 94612, US

(21) Appl. No.: 12/315,280

(22) Filed: Dec. 1, 2008

(51) Int. Cl. A01H 5/00

(52) U.S. Cl. PLT/462

(57) ABSTRACT

A new and distinct cultivar of Ornithogalum thyrsoides Jacq. named ‘Damascus’ originated as a controlled cross between the commercial cultivar Ornithogalum thyrsoides Jacq. ‘Snowflake’ and an unnamed Ornithogalum thyrsoides Jacq. cultivar. The cultivar ‘Damascus’ has R.H.S. 155 A (white group) petals at maturity and is distinguished from other similar cultivars of which the inventor is aware by its erect racemes, wide leaves that lay flat and its suitability for growth in pots.

### BOTANICAL CLASSIFICATION

Ornithogalum thyrsoides Jasq. ‘Damascus’

### VARIETY DENOMINATION

‘Damascus’

### BACKGROUND OF THE INVENTION

This invention relates to a new and distinct cultivar of the Lilaceae family. The botanical name of the plant is Ornithogalum thyrsoides Jasq. ‘Damascus’.

The new cultivar originated as a seedling from a controlled cross between the commercial variety Ornithogalum thyrsoides ‘Snowflake’ and an unnamed Ornithogalum thyrsoides. ‘Snowflake’ was the seed parent. ‘Snowflake’ is the progeny of the commercial variety ‘Bock Bay’.

The variety ‘Damascus’ is more like its pollen parent.

The new variety was first noticed, because of its erect, but relatively short racemes and its smaller flowers.

The new cultivar ‘Damascus’ has been asexually reproduced through three generations under controlled environmental conditions at a nursery in Half Moon Bay, Calif. under the direction of the inventor with its distinguishing characteristics remaining stable.

‘Damascus’ is distinguished from other Ornithogalum thyrsoides varieties of which the inventor is aware by its relatively short, upright racemes, its less upright leaves, its suitability for growth in pots and its relatively smaller flowers.

Asexual reproduction was first accomplished when vegetative cuttings were taken from the initially selected plant. Examination of asexually reproduced, successive generations grown in a nursery at Half Moon Bay, Calif. show that the combination of characteristics as herein disclosed for ‘Damascus’ remains firmly fixed through three generations.

### DESCRIPTION OF THE DRAWINGS

The accompanying drawings consist of color photographs that show the typical plant form, including the inflorescence, foliage, and sepals.

FIG. 1 is a view of the new variety showing its form, upright growth habit, dark green foliage, inflorescence, and non-pigmented petals.

FIG. 2 is a view of the inflorescence of the new variety.

FIG. 3 is a close-up view of the inflorescence of the new variety, showing opened florets, and opening florets.

FIG. 4 is a close-up view of the inflorescence of the new variety, showing opened florets, and opening florets.

FIG. 5 is a top view of a number of inflorescences of the new variety.

FIG. 6 is a view of the new variety.

FIG. 7 is a view of the base of the new variety.

FIG. 8 is a view of the upper portion of multiple inflorescences of the new variety.

FIG. 9 is a close-up, side view of florets of the new variety.

FIG. 10 is a close-up, side view of florets of the new variety.

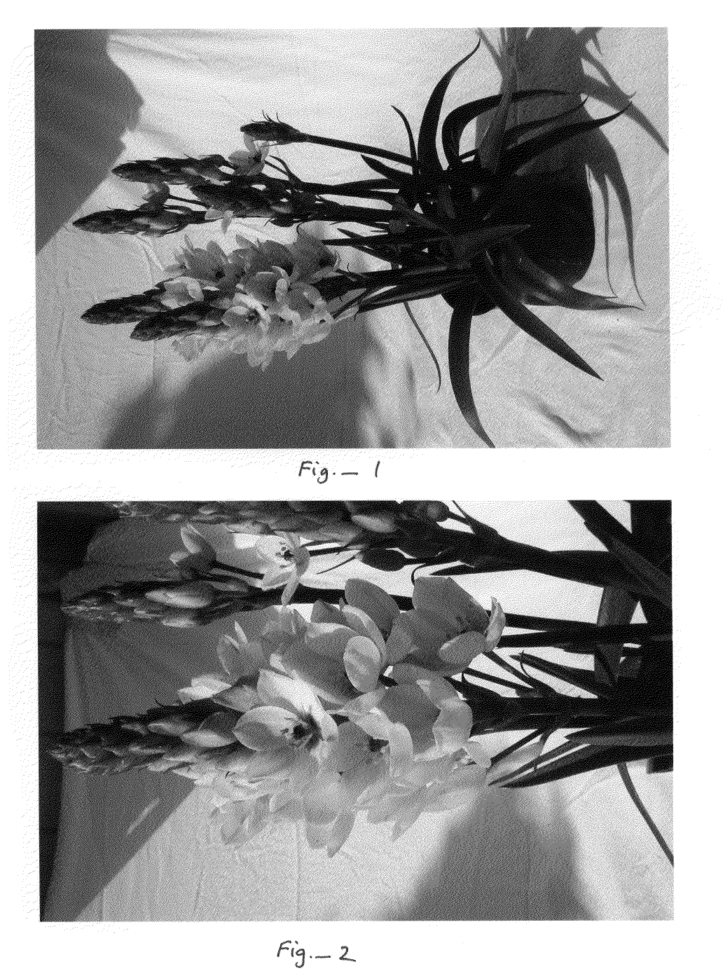

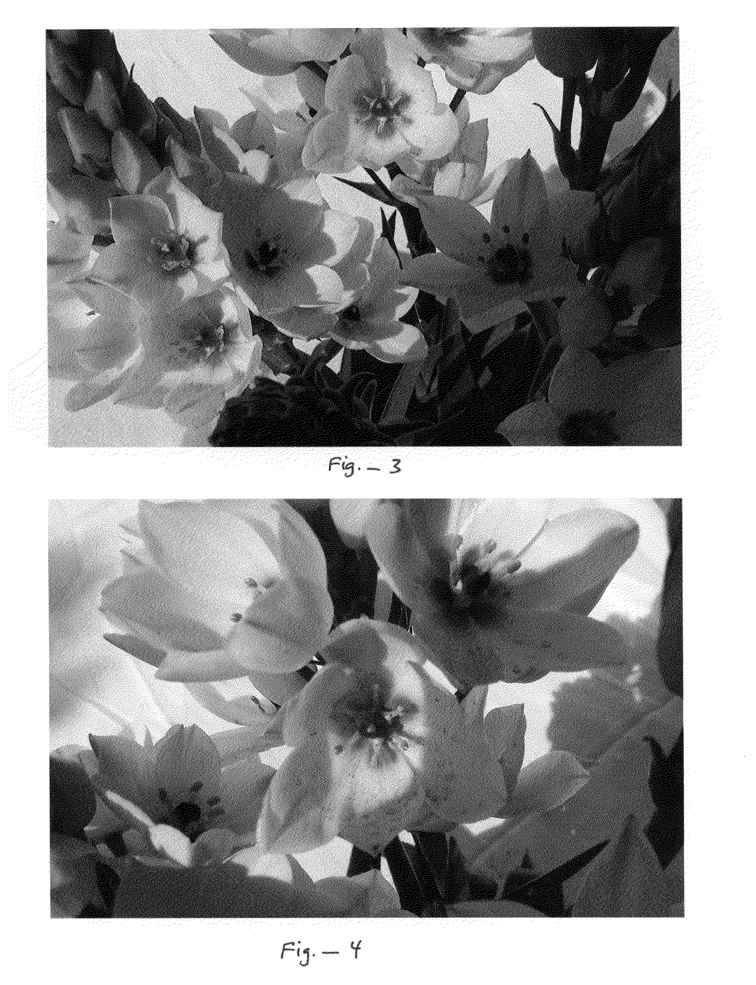

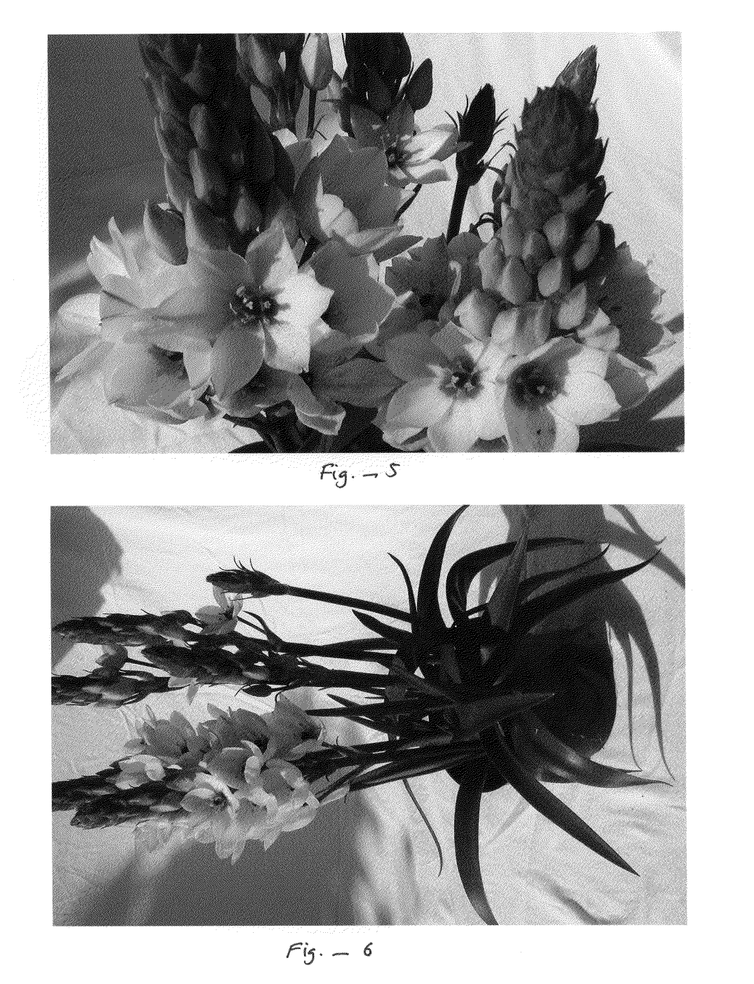

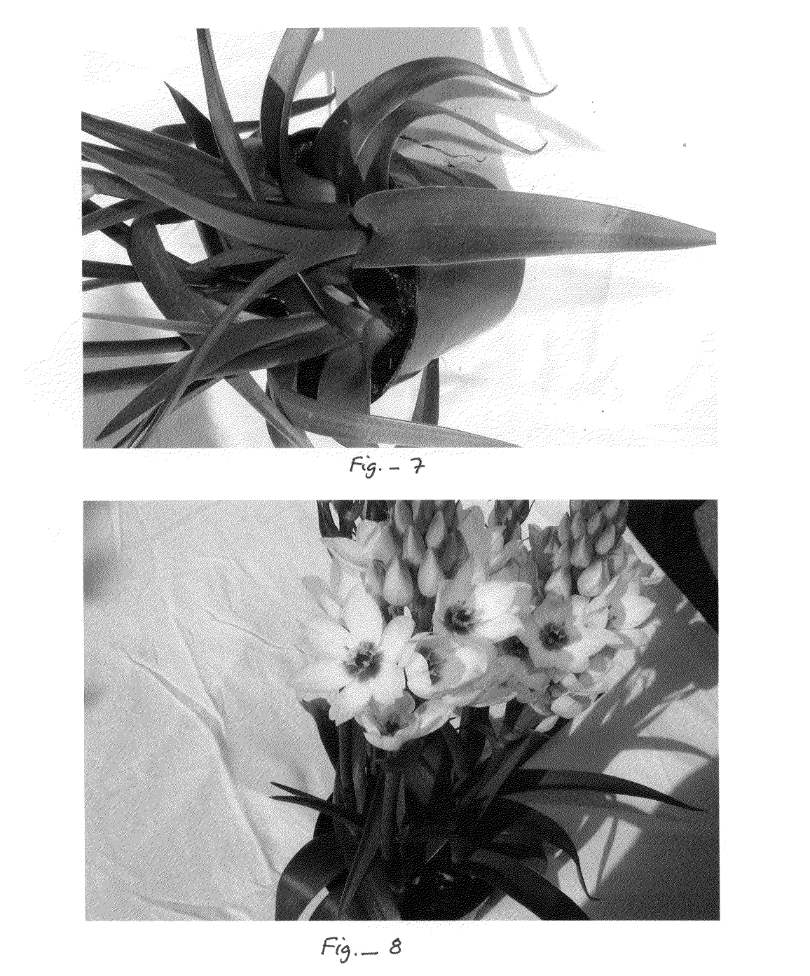

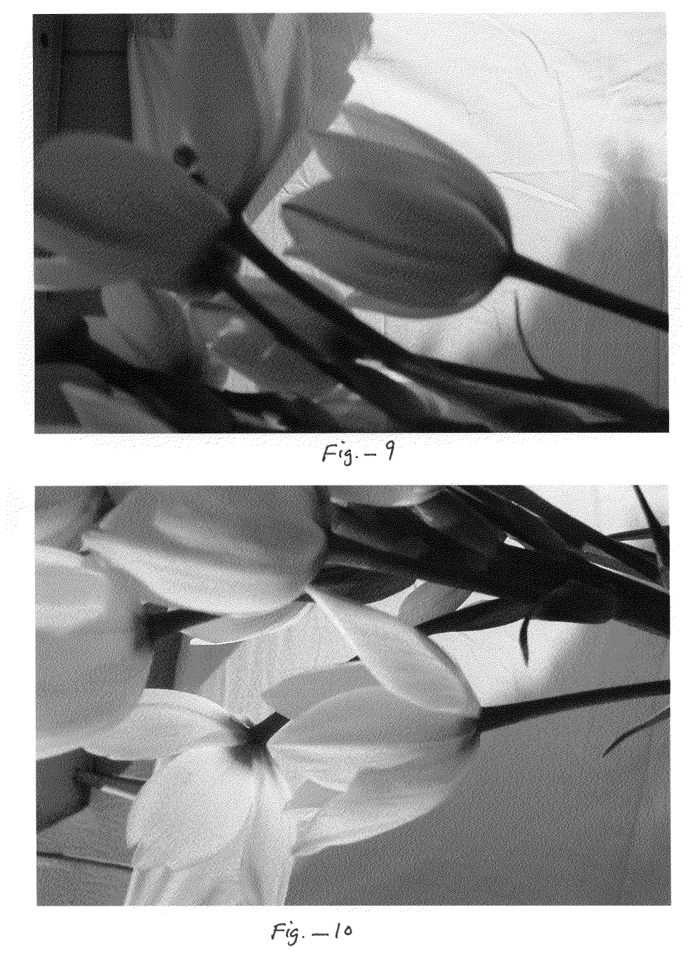

### DESCRIPTION OF THE NEW PLANT

The plants shown in the figures were grown in 6″ pots and were approximately 7 months old. The plants were started in the green house for 20 to 25 weeks and then move outside.

‘Damascus’ has not been observed under all possible environmental conditions. The phenotype may vary significantly with variations in environment such as temperature, light intensity and day length. Color determinations were made with The Royal Horticultural Society (R.H.S.) Colour Chart.
The plant:
   Origin.—Controlled cross.
   Parents.—Ornithogalum thyrsoides Jasq. ‘Snowflake’ and an unnamed Ornithogalum thyrsoides Jasq.
   Botanic classification.—Ornithogalum thyrsoides Jasq. ‘Damascus’.
   Form.—Spreading basal leaves with upright inflorescences on tall rachis. A typical plant with a mature inflorescence that is ready for sale is approximately 40 cm high when grown in a 6″ pot with appropriate soil amendments.
   Growth.—Upright rachis with spreading leaves. Inflorescence is large and showy.
   Rachis.—The rachis is upright and glaucous. Its color is R.H.S. 146 B (yellow-green group) (younger) to R.H.S. 147 B (yellow-green group).
   Width.—7 mm.
   Foliage.—Usually five leaves on a mature plant ready for commercial sale. Leaves are whorled at base of stem.
   Shape of leaf.—Fleshy, varying from somewhat channeled to flat, lanceolate. Margins are entire.
   Texture.—Glaucescent.
   Color.—Mature leaves are R.H.S. 147 B (yellow-green group). Large leaves are 27 cm long and 5 cm wide.
Inflorescence:
   Form.—Corymbose racemes. Indeterminate As many as 100 or more individual flowers (florets) per inflorescence on pedicels that elongate as the florets opens, and can be as long as 50 mm. Flowers do not produce a fragrance. The rachis for the inflorescence is strong and upright. The pedicels are predominately green — R.H.S. 146 B (yellow-green group) at maturity.
   Size of inflorescence.—Large. A typical inflorescence shown in the figures can be 17.5 cm high.
   Shape.—Basal florets opening first with pedicels that elongate that give the raceme a corymbose appearance as the inflorescence ages.
   Appearance.—Showy.
Florets:
   General.—The florets at the base of the inflorescence open first, and open more and more as the inflorescence ages. Florets are perfect and complete. Perianth is not keeled. Perianth can be hightly reflexed. Each floret is associated with a bract that can be as high as 50 mm and is same color as leaves — R.H.S. 147 B (yellow-green group).
   Corolla.—There are 6 petals that are spotted at their base — R.H.S. 152 B (yellow-green group), but predominately white — R.H.S. 155 A (white group) with the mid-vein showing on the abaxial surface — R.H.S. 145 A (yellow-green group).
   Flower diameter.—65 mm. Petals are typically 28 mm high, 18 mm wide, 4 mm wide at their attachment, and the spotted base is approximately 5 mm high and extends the width of the petal at the base.
   Stamens.—6 stamens (two sets of 3). Pollen is R.H.S. 14 A (orange group). Plant produces abundant pollen. Filament is flattened with alternate stamens having enlarged translucent wings. Filament is 3 mm wide at base and 10 mm high. Filaments with wings are 3 mm at their widest point. Anther is 2 mm high and basally attached.
   Stigma.—Generally, a three-pronged stigma on one pistil per floret. The style is 3 mm long. The stigma is approximately 2 mm long.
   Ovary.—The ovary is three-celled with axile placentae. The base of the ovary is R.H.S. 152 A (yellow-green group) and the top of the ovary is R.H.S. 166 A (greyed-orange group). The ovary is 6 mm high and has a diameter of 7 mm.

I claim:

1. A new and distinct Ornithogalum plant named ‘Damascus’ substantially as herein shown and described.
